(19) United States
(12) Patent Application Publication
(10) Pub. No.: US 2017/0184015 A1
(43) Pub. Date: Jun. 29, 2017

(54) BIOFUEL POWER GENERATION SYSTEMS

(71) Applicant: Kohler Co., Kohler, WI (US)

(72) Inventors: Travis J. Andren, Sheboygan, WI (US); Manuel Rumao, Sheboygan, WI (US); Eric B. Hudak, Sheboygan, WI (US); Clayton C. Garrels, Sheboygan, WI (US)

(21) Appl. No.: 15/301,997

(22) PCT Filed: Dec. 30, 2014
(86) PCT No.: PCT/US14/72725

§ 371 Date: Oct. 5, 2016

(51) Int. Cl. F02B 43/08; C12M 1/107; H02K 7/18; B01J 19/24; F02M 21/02; F02B 63/04; C12M 1/00; C12M 3/00

(52) U.S. Cl. CPC F02B 43/08; C12M 43/08; C12M 21/04; C12M 23/44; C12M 23/36; B01J 19/24; F02M 21/0215; F02B 63/04; H02K 7/1815; B01J 2219/24; B01J 2219/00049

(57) ABSTRACT

An integrated modular biomass to electric power generation system is disclosed. The system comprises various subsystem modules including components which may be prefabricated and mounted on separate and portable self-supporting liftable platforms in a factory controlled environment. The modules may include biomass to biofuel conversion and refinement process related modules and power generation modules. The modules may be transported from the factory to a power generation site, which in some non-limiting examples may comprise part of an agricultural or farming site that produces animal and/or agricultural wastes which may be converted to biofuel. The modules are positioned on site and fluidly coupled together to form a complete biofuel powered electric generation system. A plurality of standardized size subsystem modules of several different process or electrical capacities may be provided for selection to form the complete system. The system may include a biogas production process, biodiesel production process, or both.

### BACKGROUND OF THE INVENTION

Biological material or “biomass” may be converted into various combustible forms of biofuel, including biogas and biodiesel. Biogas (primarily methane) can be produced via the anaerobic digestion of plant and animal waste materials and other organics in a digester, or as part of the sewage treatment process. This processes primarily produces methane and carbon dioxide. Methane can be used for heating, cooking or the generation of electricity via a generator coupled to an internal combustion engine. Biodiesel, a liquid form of biofuel, may similarly be burned in an internal combustion engine-generator set for producing electric power.

### SUMMARY OF THE INVENTION

A power generation system is disclosed that integrates the design, construction and manufacture of a complete biofuel electric generating plant intended to achieve low-cost and scale-ability. The power generation system includes various subsystems and components which are integrated together to create a system for conversion of biomass to electricity. The system may include provisions for converting biomass into biogas and/or biodiesel into a single system. In one configuration, the individual subsystems for convening the biomass into biofuel, biofuel conditioning, power generation, and others may be modularized and pre-manufactured for assembly into a complete power generation package. These modular subsystems may be mounted on single support platform in some arrangements which is transportable as a single unit.

In one implementation, a modular integrated biofuel power generation system includes a digester subsystem module including a digester configured to convert biomass into biogas, and an electric power generation subsystem module including an internal combustion engine and electrical generator configured for generating electricity. The internal combustion engine fluidly may be coupled to and configured to burn the biogas produced by the digester. The components of each module may be mourned on individual self-supporting and transportable structural platforms. The modules are fluidly coupled together on a power generation site after placement of each module.

In another implementation, a modular integrated biofuel power generation system includes a filter/treatment subsystem module configured to convert raw oils and fats into biodiesel via a chemical reaction, a cleaner/refining subsystem module configured to purify the biodiesel by removing byproducts of the chemical reaction, and a power generation subsystem module including an internal combustion engine and electrical generator configured for generating electricity. The internal combustion engine may be fluidly coupled to and configured to burn the biodiesel produced by the system. The components of each module may be mounted on an individual self-supporting and transportable structural platforms. The modules may be fluidly coupled together on a power generation site after placement of each module.

A method for forming a modular integrated biofuel power generation system is provided. The method includes the steps of selecting a factory prefabricated self-supporting first digester subsystem module having a first capacity from a plurality of prefabricated self-supporting digester subsystem modules each having a different capacity, the first digester subsystem module including a digester configured to convert biomass into biogas, wherein the first digester subsystem module comprises a transportable structural equipment platform configured to be lifted as a single unit; selecting a factory prefabricated self-supporting first biogas treatment subsystem module having a first capacity from a plurality of prefabricated self-supporting biogas treatment subsystem modules each having a different capacity, the first biogas treatment subsystem module configured to refine the biogas produced by the digester, wherein the first biogas treatment subsystem module comprises a transportable structural equipment platform configured to be lifted as a single unit; selecting a factory prefabricated self-supporting first electric power generation subsystem module having a first electrical output rating from a plurality of prefabricated self-supporting power generation subsystem modules each having a different electrical output rating, the first electric power generation subsystem module including an internal combustion engine and electrical generator configured for generating electricity, wherein the first power generation subsystem module comprises a transportable structural equipment platform configured to be lifted as a single unit; transporting the first digester subsystem module, first biogas treatment subsystem module, and first electric power generation subsystem module from a factory to a remote power generation site location; emplacing the first digester subsystem module, first biogas treatment subsystem module, and first electric power generation subsystem module at the power generation site; fluidly coupling the digester to the first biogas treatment subsystem module via a field run flow conduit; and fluidly coupling the internal combustion engine to the first biogas treatment subsystem module via a field run flow conduit. In some implementations, a waste heat recovery system configured to capture heat generated by the internal combustion engine of the power generation subsystem module may be provided. In such systems, the method may further include installing an insulated flow loop or path at the power generation site configured to transfer a heated working fluid from the first power generation subsystem module to the first digester subsystem module for heating the digester.

### BRIEF DESCRIPTION OF THE DRAWINGS

The features of exemplary apparatus and systems will he described with reference to the following drawings, where like elements are labeled similarly, and in which:

FIG. 1 is a schematic diagram of an exemplary integrated biofuel power generation system including process equipment for converting biomass to biofuel and burning the biofuel in an internal combustion engine coupled to an electric power generator for producing electricity;

FIG. 2 is a schematic process flow diagram of an example biogas power generation process;

FIG. 3 is a schematic process flow diagram of an example biogas power generation process showing provisions for dual use of the biogas produced;

FIG. 4 is a schematic process flow diagram of an example biodiesel power generation process;

FIG. 5 is a schematic drawing of an exemplary integrated biofuel power generation system including process equipment for converting biomass to biofuel and burning the biofuel in an internal combustion engine coupled to an electric power generator for producing electricity;

FIG. 6 is a schematic process flow diagram of an example dual biofuel power generation process comprising provisions for producing biogas and biodiesel, and an internal combustion engine configured for burning either fuel type;

FIG. 7 shows example composting toilet systems which produces biogas and fertilizer;

FIG. 8 shows an example rocket-type torrefaction toilet system;

FIG. 9 shows an example microwave torrefaction toilet system;

FIG. 10 is a perspective view of a prefabricated self-supporting, liftable, and transportable power generation subsystem module;

FIG. 11 is schematic diagram showing a factory kit comprising a plurality of different capacity prefabricated biofuel conversion-power generation subsystem modules which may be selected to form a complete biomass-to-electric power generation system; and

FIG. 12 is a schematic diagram showing a waste heat recovery system configured to capture and transfer waste produced by the power generation module to a digester.

All drawings are schematic and not necessarily to scale.

### DETAILED DESCRIPTION OF EMBODIMENTS

The present invention generally relates to biofuel powered systems for producing electric power, and more particularly to a system including components for convening biomass into biofuel and combusting the biofuel to generate electric power in a single integrated system.

The features and benefits of the present disclosure are illustrated and described herein by reference to exemplary apparatuses and systems. This description of these examples is intended to be read in connection with the accompanying drawings, which are to be considered pan of the entire written description. Accordingly, the present disclosure expressly should not be limited to such examples illustrating some possible non-limiting combination of features that may exist alone or in other combinations of features; the scope of the claimed invention being defined by the claims appended hereto.

In the description of examples disclosed herein, any reference to direction or orientation is merely intended for convenience of description and is not intended in any way to limit the scope of the present invention. Relative terms such as “lower,” “upper,” “horizontal,” “vertical,”, “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation. Terms such as “attached,” “coupled,” “affixed,” “connected,” “interconnected,” and the like refer to a relationship wherein structures are secured or attached to one another either directly or indirectly through intervening structures, as well as both movable or rigid attachments or relationships, unless expressly described otherwise.

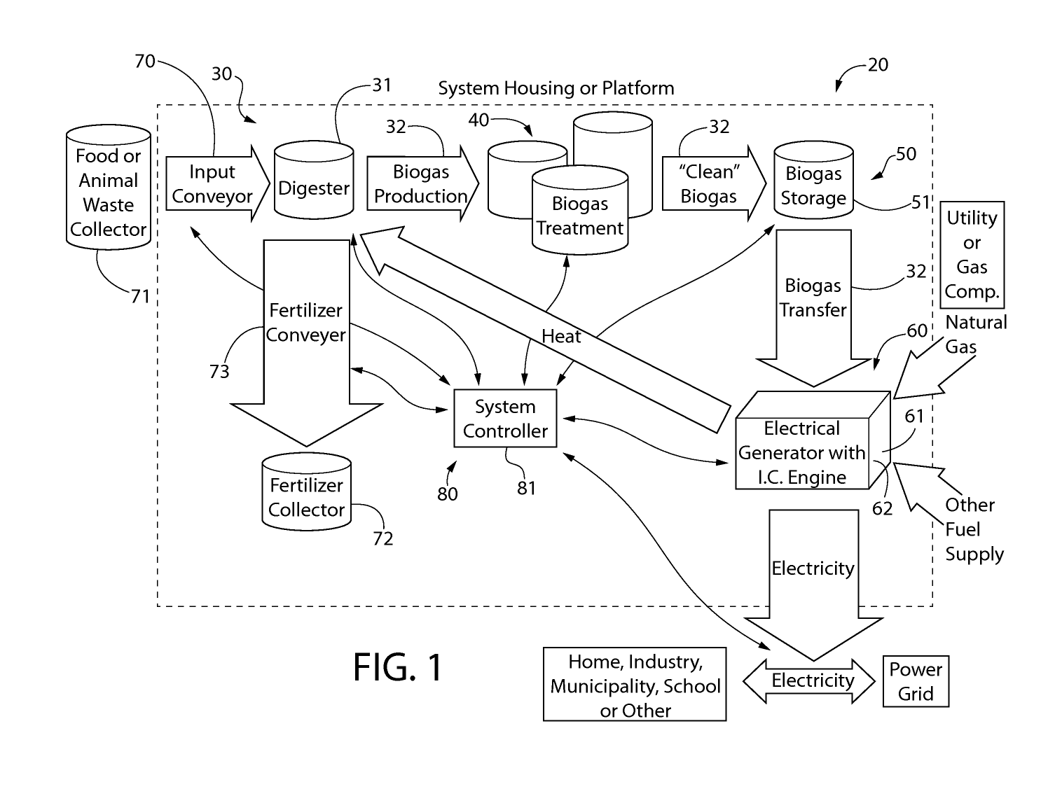

FIG. 1 depicts an example biofuel power generation system 20. In this non limiting example, the system is configured for producing biogas. However, it should be noted that the system is not limited in this regard. In other configurations, the system may be configured to produce biodiesel or other fuels.

Power generation system 20 may generally include one or more of a digester subsystem 30 including a digester 31 which converts biomass into biogas, a biogas treatment subsystem 40 including biogas treatment equipment, a biogas storage subsystem 50 including a biogas storage vessel 51, and an electric generation subsystem 60 including an internal combustion engine 61 and electrical generator 62 configured for convening mechanical energy from the engine into electric current. The engine 61 may burn biogas or other fuels. Waste heat produced by the engine 61 may be captured and supplied to the digester 31 to enhance the anaerobic digestion process. The waste heat may comprise heated exhaust combustion gas from burning the biogas and/or ambient waste heat radiated from the engine itself into the ambient environment within an enclosed engine housing or module. The waste heat may be routed to the digester 31 via insulated flow conduits such as air ducts or coolant piping.

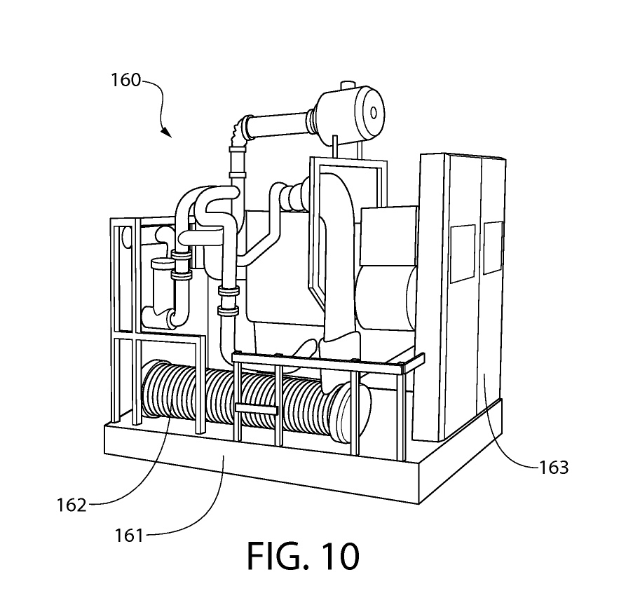

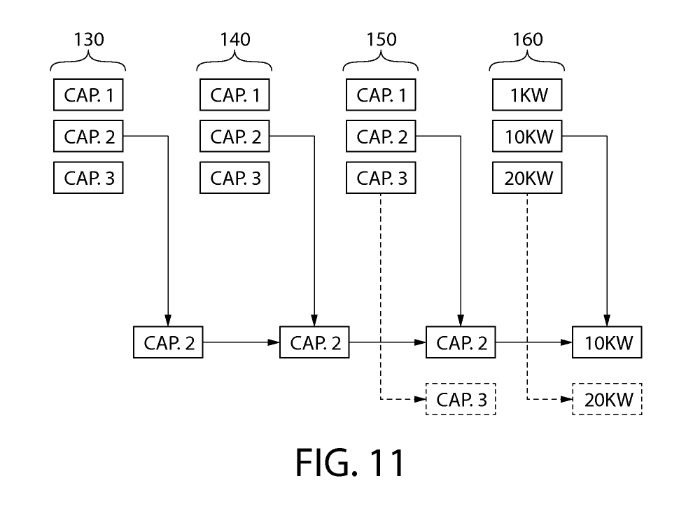

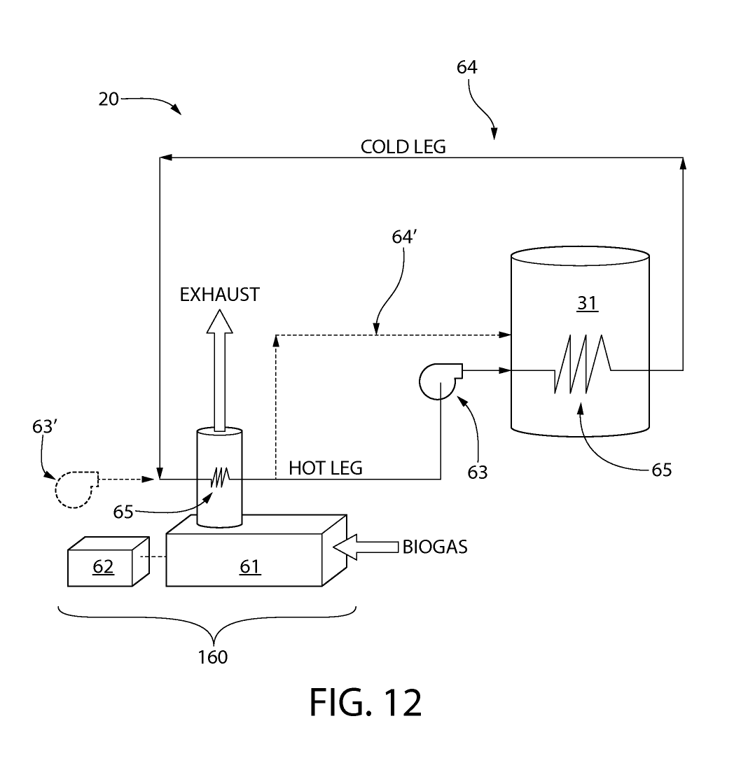

FIG. 12 is a schematic diagram showing non-limiting examples of a waste heat recovery system for capturing heat generated by the engine 61 to supplement the heat in the enclosed digester 31. The waste heat recovery system comprises a working fluid (i.e. a liquid, air, or gas) which is heated by the waste heat from the engine 61 and transferred to the digester 31. The waste heat recovery system may he a recirculating liquid coolant type system (solid lines) or a once-through hot air type system (dashed lines) in exemplary non-limiting implementations. A recirculating liquid coolant system may include one or more circulating pumps 63 which circulate a coolant through a closed flow loop 64 between the engine 61 (e.g. power generation module 160 in FIGS. 10 and 11) and digester 31 tank. In this system, the source of waste heat captured may be heated combustion gases produced by the engine 61. Any suitable coolant may be used, including for example without limitation water glycol, or another. Heat exchanger coils 65 may be disposed in the exhaust stack as shown which extracts heat from the combustion gases and heats the coolant. Coils 65 may be similarly disposed in the digester 31 tank which transfers heat to the enclosed environment within the digester tank, thereby cooling the coolant. The heat exchanger coils 65 (e.g. tubing) may be formed of a good heat conducting material such as copper, aluminum, steel, or other metals used in such applications. In some embodiments, the coils may include fins to maximize the heat transfer area. The coolant circulates through the closed flow loop 64 as shown between the hot and cold legs of the loop. The flow loop 64 may be formed by suitable insulated piping and/or tubing, Suitable flow control and monitoring devices (e.g. valving, orifices, pressure regulators, gauges, temperature monitoring equipment, etc.) may be provided to complete the system.

A once-through hot air type system (dashed lines in FIG. 12) may include a blower 63′ which suctions and discharges ambient air through coils 65 disposed in the engine exhaust stack. The air is heated by the combustion gases and may flow directly into the enclosed digester 31 tank environment, where the hot air is discharged. In some implementations, the hot air may instead flow through coils 65 in the digester 31 if it is desired to isolate the hot air from the digester environment. Any suitable insulated air flow ducts or conduit may be used to construct the once-through flow path 64′ including metal ductwork, piping, and/or tubing. Similar liquid coolant or hot air systems may be used to capture non-combustion ambient waste heat rejected by the engine itself.

The digester 31 may receive biomass, such, as food or animal waste or energy crops. The digester 31 may be an anaerobic digester. The waste may be stored in a sealed tank and/or treated in various ways. The waste may be transformed in the digester 31 to generate biogas, such as methane (CH₄) and carbon-dioxide (CO₂) in addition to some trace gases such as water vapor, hydrogen sulfide (H₂S), nitrogen, hydrogen and oxygen.

The biogas produced in the digester 31 may be conveyed by tubular conduits 32 (as discussed below) to the biogas treatment subsystem 40. The biogas treatment subsystem 40 may refine and remove one or more byproduct components from the biogas, such as carbon dioxide, water vapor, and the trace gases such as H₂S, before the biogas is used downstream in the process. The removal of these components may be beneficial, as H₂S gas is corrosive and the water vapor may cause corrosion when combined with H₂S forming an acid on metal surfaces.

The digester 31 and biogas treatment substation 40 may generate biogas at various rates. In some systems, biogas may not be generated consistently at a rate sufficient to continuously run the electric generation subsystem 60. In these and other systems, the refined biogas may be stored in the biogas storage subsystem vessel 51 or other biogas storage 50 units. The biogas storage subsystem vessel 51 may include a compressor to keep the biogas stored under pressure in the storage vessel 51. The stored biogas may be transferred when desired to the electric generation subsystem 60 and used as fuel to run the engine 61.

In other systems, the storage vessel 51 may be storage tanks or units of a size capable of being transported and/or used with portable electric generation subsystems 60, remotely from the rest of the system 20. In some of these systems, the storage vessel 51 may be replaceable and quickly connected with the conduits 32 to provide for the refilling of many storage vessels 51 using the same system 20. Other variations are possible.

In some systems, the engine 61 or electric generation subsystem 60 may additionally be selectively connected with one or more additional fuel sources or fuel lines. For example, natural gas (or propane) from a municipal utility may be provided to the electric generation subsystem 60 to augment the engine fuel needs beyond the production rate of biogas. As another example, the electric generation subsystem 60 may be connected with a portable or permanent fuel tank or other fuel supply, which may provide fuel (such as diesel, gasoline, natural gas, propane, methane, or other fuels) to the engine 61 in addition to (or to supplement or augment) the biogas. In other systems, the electric generation subsystem 60 may not be connected with one or more fuel lines provided by a utility or gas company or other fuel tanks. Other variations are possible.

Some systems may additionally include a biomass furnace, which may provide additional externally supplied heat to the digester subsystem 30 for increasing the anaerobic reaction rate. The biomass furnace may be used to burn combustible biomass materials such as wood or other organic materials. Heat from the burned biomass materials may be provided to, and used to heat, the digester 31.

In some configurations, the generator 62 may additionally include or alternatively be an alternator. The alternator may be an electromechanical device operably coupled with the engine 61. The alternator may include or use a rotating magnetic field with a stationary armature, a rotating armature with a stationary magnetic field, or a linear alternator. For example, the engine 61 may produce mechanical energy when operated which the alternator converts into electrical energy, such as alternating current.

The power generation system 20 may further include a fuel input conveyor 70 for feeding biomass (e.g. plant material, food waste, and/or animal waste) from a fuel collector vessel 71 to the digester 31 or other component of the digester subsystem 30. The biomass may be supplied to and/or stored in the fuel collector vessel 71 until processed in the system. The conveyor 70 may be any suitable type and configuration of a conveyor depending on the nature of the biomass (e.g. solid or liquid). Some non-limiting examples include roller conveyors with recirculating belts or grates, augers, tubular conveyors such as pressured or vacuum flow conduits (e.g. piping), and others. The type of conveyor used may primarily be dictated by the nature and form of the biomass material.

The power generation system 20 may further include a fertilizer collector vessel 72 and fertilizer conveyor 73 configured to transfer waste material from the digester 31 to the vessel. The conveyor 73 may be any suitable type and configuration of a conveyor, including for example without limitation roller conveyors, augers, or tubular flow conduits depending on the nature (e.g. solid or liquid) of the digester waste. In addition to producing biogas, the digester 31 may produce a high quality fertilizer as a byproduct. Solid and liquid separation equipment may be provided to reclaim the fertilizer solids for agricultural uses. In some instances, an automated fertilizer collector and packaging device may be provided such as a bagger disposed at the end of the fertilizer conveyor 73 or directly beneath the digester 31.

The biogas produced in the digester 31 may be conveyed between the various subsystems in power generation system 20 by tubular conduits 32 such as piping and/or tubing. The conduits 32 may therefore be used to interconnect one or more of the digester subsystem 30, biogas treatment subsystem 40, a biogas storage subsystem 50, and electric generation subsystem 60 in the process stream.

Operation of the power generation system 20 may be controlled by an appropriately configured electronic central control subsystem 80 including a computerized processor-based controller 81 in communication with the various foregoing subsystems 30, 40, 50, 60, the fuel input conveyor 70, and the fertilizer conveyor 73. Controller 81 may further control the dispatch of electricity produced by the electric generation subsystem 60 to the power grid or locally for on-site uses proximate to the power generation system 20. In alternative or additional systems, one or more distributed control systems (DCS) may be provided in addition to or instead of central control subsystem 80 to control operation of one or more of the subsystems or conveyors. Accordingly, the configuration and type of control system used to coordinate and control operation of the entire power generation system 20 is not limited to any particular type of control strategy or equipment arrangement.

The central control subsystem 80 may include some or all components and appurtenance for providing a fully functional control system. For example, the central control subsystem 80 may include sensors, relays, and other components operable to monitor important system operating parameters and control the operation of the various subsystems. The central control substation may include a controller 81. The controller 81 may include all components and appurtenance for providing a fully functional system controller, such as for example without limitation one or more programmable processors, non-transitory computer readable medium containing computer-executable instructions for directing operation of the one or more processors, volatile and non-volatile memory, non-transitory computer readable medium permanent and removable data storage devices (e.g. hard disk, solid state drives, USB drives, etc.), wired and/or wireless communication links, input/output devices and interfaces, visual display, etc. When performed by processor, the computer-executable instructions cause the processor to monitor and control operation of the biofuel power generation system 20 and its various subsystems 30, 40, 50, 60. The non-transitory medium could be a disk, solid state drive, a tape, a chip, or a random access memory as some non-limiting examples.

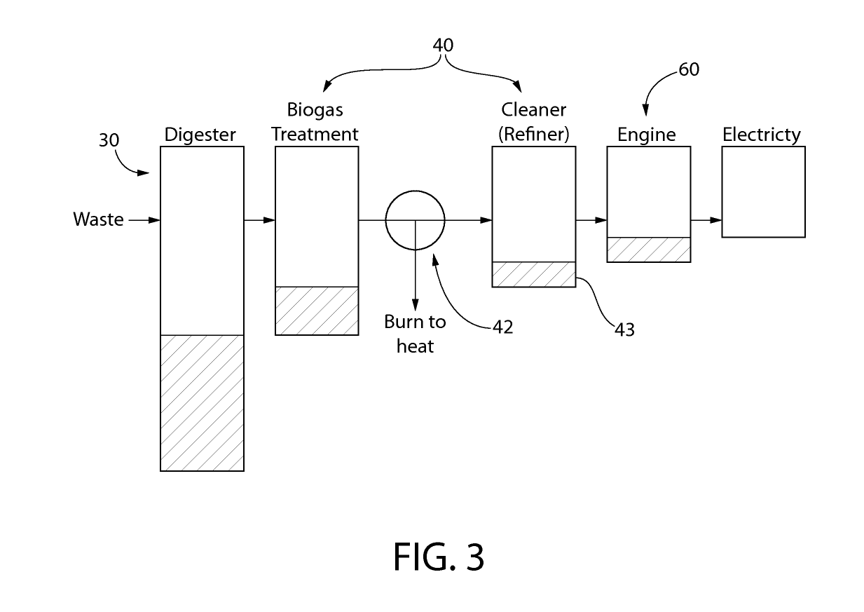

The central control subsystem 80 may be configured to monitor one or more parameters such as: biomass material input level (weight, height, density, composition); digester temperature, pressure, humidity, time in digester, digester waste level, etc.; amount of fertilizer produces; biogas temperature, pressure, flow rate, etc.; biogas treatment system “consumable level,” temperature, pressure, flow rate, etc.; clean methane gas temperature, pressure, flow rates, and chemical makeup; storage vessel temperature, pressure, etc.; engine parameters (speed, load, etc.); generator parameters; electrical load; and others. The central control subsystem 80 may control such operations as: input conveyor (on, off, speed, etc.); stirrer in the digester; digester heating (for biogas system); fertilizer conveyor from digester; valve for biogas output (open/close); three-way valve if provided for directing flow of biogas output from biogas treatment (e.g. for heating/cooking or power generation in FIG. 3); output to the power generation system; typical engine/generator controls; load production/rate of engine or generator; electricity delivered to the grid or a load; heat transferred back to the digester e.g. from the engine); and others.

The controller 81 may, for example, monitor a temperature of the digester 31. The controller Si may control an amount of heat transferred to the digester 31 to maintain the temperature of the digester 31 above a temperature threshold or within a temperature range. For example, heat from the electric generation subsystem 60 may be passed to the digester in various ways and through various channels as shown in FIGS. 1 and 12, and the controller 81 may control the channels to control the temperature of the digester 31. As another example, the controller 81 may control additional heating devices (or fuel or electricity provided to additional heating devices) such as the biomass furnace, which may provide heat to the digester 31, thereby controlling a temperature of the digester.

The controller 81 may control one or more valves fm distributing biogas within the system 20. For example, the controller 81 may monitor a biogas production within the digester 31, and may maintain the digester 31 in a sealed environment until the generated biogas (or percentage of biogas) has exceeded a threshold, at which time the controller 81 may activate a valve or otherwise control the passing of the biogas to the biogas treatment subsystem 40. The controller 81 may similarly monitor the treated biogas within the biogas treatment subsystem 40, and may activate a valve or otherwise control the passing of the biogas to the biogas storage 50.

The controller 81 may monitor a state of the digestion occurring within the digester 31. For example, the controller 81 may monitor various parameters of the digester and biomass, including a composition of the biomass, a weight of the biomass, a volume of the biomass, and/or a mass of the biomass. The controller 81 may store, access from a remote location, or learn and store algorithms or ratios of one or more parameters of the digester and biomass which correspond to various stages of digestion. For example, the controller 81 may store thresholds or algorithm outputs which may indicate that the existence of a chemical compound exceeding a certain threshold percentage indicates that digestion has reached a point at which biogas may be transferred to the biogas treatment subsystem 40. As another example, the controller 81 may store thresholds or algorithm outputs which may indicate that at a certain ratio of weight to volume, the digestion has reached a point at which biogas may be transferred to the biogas treatment subsystem 40. The controller 81 may monitor the parameters of the digester 31, and may control the transfer of biogas from the digester 31 when the parameters meet the stored thresholds or algorithms.

The controller 81 may control the operation of the electric generation subsystem 60 and the transfer of the biogas from the biogas storage 50 to the engine 61. The controller 81 may store or determine a biogas threshold which may indicate an amount of biogas necessary to operate the engine 61 for a given amount of time, such as for a period of time which exceeds a threshold or for a period of time which will generate enough electricity to make the system achieve a given efficiency. The controller 81 may control a valve or components necessary to transfer the biogas from the biogas storage 50 to the engine 61 according to the stored or determined biogas thresholds.

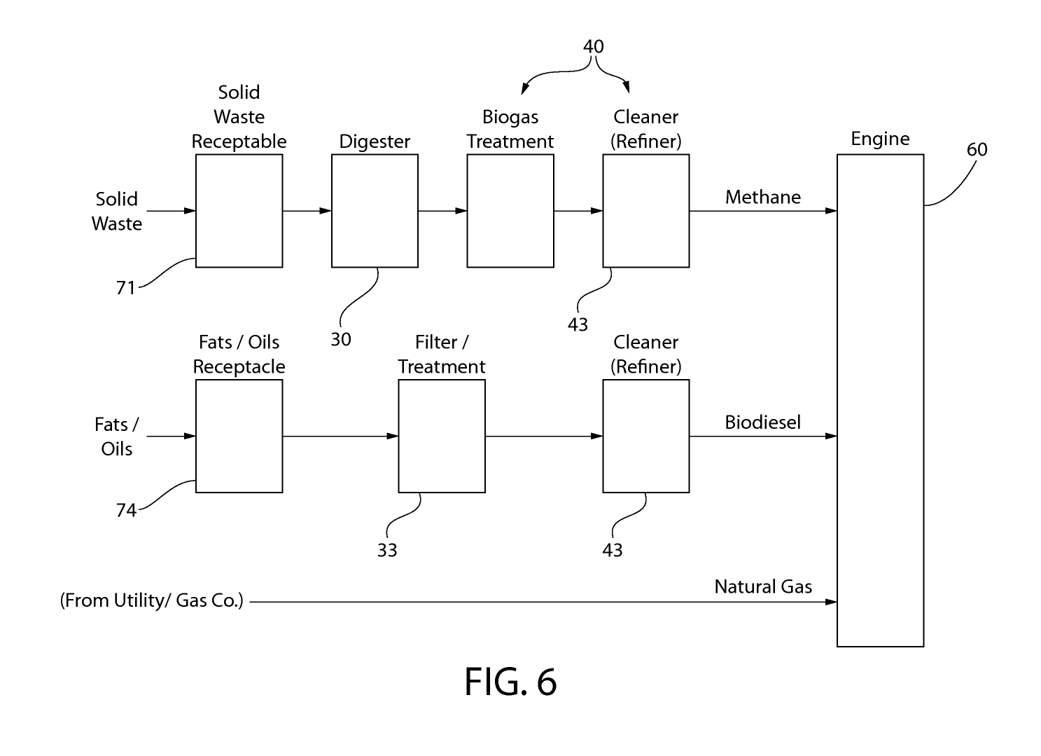

The controller 81 may control a source of fuel to be supplied to the engine 61. For example, in a system as shown in FIG. 6, the controller may control one or more valves, and/or one or more engine components, to enable the engine 61 to receive and operate using methane, biodiesel, or natural gas.

The controller 81 may receive one or more user inputs, and may control the system in accordance with user inputs. For example, the controller 81 may receive an input through a user interface, which may indicate that the user does (or does not) want to use the generated biogas for something other than powering the engine 61. The user may, for example, want to use the biogas for cooking, and may provide an input to the controller 81 indicating as much. The controller 81 may control one or more valves or components to direct the biogas to the alternative location or device, for use as desired by the user.

The controller 81 may control a power output of the electric generation subsystem. For example, the controller 81 may control one or more aspects of the engine, such as engine speed, which may control or affect the output power from the subsystem 60. The controller may perform various power generation system tasks or functions, such as connecting loads to the electric. generation subsystem 60, paralleling the electric generation subsystem 60 with other power generation systems or power grids, shedding loads from the system, determining when utility power is or is not available, and other functions.

The controller 81 may track parameters over time and may adjust one or more control functions as a result of the tracked data. This may prove particularly useful as the integrity of components in the system change over time. For example, the controller 81 may track an efficiency of one or more components of the system 20, and may adjust the operating parameters of the system to maximize the efficiency of one or more components. As a specific example, the controller 81 may monitor the efficiency or speed of the digester 31 over a range of temperatures or fill-levels. The controller 81 may update parameters such as conveyor rate or heat provided to the digester 31 to achieve the most efficient operation of the digester. As another specific example, the controller 81 may control engine operating parameters (such as biogas fuel flow), some of which may change over time as the engine is run. Accordingly, the controller 81 may update thresholds and operating parameters to optimize aspects of the engine (such as output, run time, etc.).

The controller 81 may communicate any or all of the data it tracks, operating parameters of any component of the system, or any other data or information to a display device, a server, a user device, or any other device or component. Such communication may be performed through a wired or wireless network (or both). For example, all information from the controller 81 may be sent to and stored on a remote server. The data may be accessible to other users, consumers, technicians, manufacturers, and others from the server. In some instances, a user may use a mobile device or smart phone to log into the server and monitor the status of the system 20. Other variations are possible.

The controller 81 may monitor the status and/or integrity of one or more components or subsystems of the system 20. The controller 81 may compare the status or integrity of the components to known (or learned) thresholds or parameters to determine when maintenance is due or a component appears likely to (or actually does) fail. The controller 81 may provide alerts or warnings when the maintenance is due or a component appears likely to (or actually does) fail. These alerts or warnings may be provided via a display connected with the controller 81 or subsystem 80, another component: of the system 20, or to one or more devices or servers via a wired or wireless network (or both).

The controller 81 may monitor the time of day and/or costs of fuel (such as natural gas costs from a utility or gas company) or power from other sources (such as utility power). The controller 81 may control the system to use the biogas generated and stored in the biogas storage 50 when the cost of using power from other sources (such as utility power) or fuel (such as natural gas from the gas company) are above a threshold or are expected to maximum. For example, the controller 81 may be programmed to recognize (or may determine based on tracked data) that the highest costs for utility power occur between 5:00 pm and 8:00 pm. The controller 81 may control the biogas storage 50 to provide fuel to the engine 61 and operate the electric generation subsystem 60 during part or all of the time between 5:00 pm and 8:00 pm. Many other variations are possible.

The controller 81 may communicate any or all of the data it tracks, operating parameters of any component of the system, or any other data or information to a display device, a server, a user device, or any other device or component. Such communication may be performed through a wired or wireless network (or both). For example, all information from the controller 81 may be sent to and stored on a remote server. The data may be accessible to other users, consumers, technicians, manufacturers, and others from the server. In some instances, a user may use a mobile device or smart phone to low into the server and monitor the status of the system 20. In some instances, calculations or determinations (such as maintenance determinations) or modifications to thresholds and logic used by the controller (such as modifying a threshold for the temperature of the digester 31) may he determined at the server, and then sent to the controller 81. Other variations are possible.

The foregoing, subsystems 30, 40, 50, 60, and components of the power generation system 20 may be modularized being each comprised of individually constructed subsystem modular units. Accordingly, the subsystem modular units may be individually pre-manufactured and pre-packaged to include pre-mounted components and substantially all ancillary equipment necessary for a fully functional subsystem (e.g. control components, instrumentation and monitoring components, power drives, electric and control wiring, piping/tubing, etc.). These components may be mounted on a common support structure (i.e. skid or structural platform). Accordingly, a subsystem modular unit so constructed may be self-supporting and handled/transported as a single complete functional unit. In one example, the platform may comprise an array of joined structural steel members of one or more shape (e.g. I-beam, channels, angles, etc.) onto which individual components/equipment are skid-mounted. The platform may be sized with a maximum length and width in some instances suitable for transport by a flatbed tractor-trailer, rail, or other.

FIG. 10 shows a non-limiting example of a modularized, skid-mounted power generation module 160 which comprises the components of electric generation subsystem 60 (e.g. engine, generator, ancillary process and control equipment, control panels 163, etc.). The equipment is mounted on a common support base or platform 161 formed of structural steel rails (e.g. I-beam sections, C-sections, angles, etc.) and similar cross-piece structural members which provide a portable base or platform for mounting the components in a rigid manner. Wiring (e.g. electrical and/or control) and flow conduits (e.g. pipe and/or tubing) are pre-installed. Both flow conduits and wiring are terminated and configured with interfaces for coupling to external flow and wiring hookups from adjoining biofuel process modules and/or other on-site equipment. In some implementations, a metal enclosure structure (not shown) may be provided which is mounted on the skid or platform to fully or partially enclose the engine, generator, and other equipment of the power generation module 160.

In lieu of components and equipment for each subsystem 30, 40, 50, 60 having to be individually transported to and assembled on-site in piece-meal fashion which is labor intensive and subject to prevailing weather conditions, the subsystem modular units advantageously may be manufactured and assembled in the controlled environment of a factory. Once shipped to the power veneration system operating site, the fully complete units may simply be located in their respective appropriate positions and interconnected via suitable field run electrical and control wiring, piping/tubing, and other interconnections to form a fully assembled power generation system 20. In some designs, the entire power generation system 20 may fit onto a single support platform which can be transported to the site. Alternatively, some of the subsystem modular units may be combined onto two or more common platforms depending on size restrictions for shipping and weight of components.

It will be appreciated that the subsystem modular units further provides the flexibility to highly customize the final power generation system 20 desired by the end user. For example, the end user would simply decide on the type of power generation system desired (e.g. biogas and/or biodiesel) and size (i.e. KW output). The appropriate type of subsystem modular units needed may then be selected from a plurality of pre-manufactured subsystem units already available in inventory and mounted onto one or more structural platforms or bases if combining some or all of the components from different subsystem is possible. Advantageously, this approach also decreases turnkey construction time resulting in delivery and erection of an operating biofuel power generation system on the end user's site in a shorter amount of time and lesser cost. In addition, if the end user desired alternate components for some of the pre-fabricated modules, it will take less time to modify an available pre-fabricated module and make equipment swaps than to build an entire module from scratch.

The modular construction of the biofuel power generation system 20 provides a pre-selected packaged kit able to assembled for a desired electric power output node size (i.e. kilowatt or KW) based on the subsystem modular units selected and used. The subsystem modular units provide sort of a “plug and play” type interchangeability via using commonly configured and sized interfaces (e.g. piping connections, electrical/control wiring connections, etc.) for each modular unit in the power generation system. This allows substitution of any of a number of different node size modular units into the final assembled power generation package either initially, or after installation. For example, if an end user initially selects and uses a 10 KW size power generation system 20, but later plans to expand their facilities which will increase the onsite power demand, the original 10 KW power generation subsystem 60 may be swapped out for a larger capacity power generation subsystem (e.g. 20 KW). During initial design of a biofuel power generation system 20, the modular unit approach also allows the selection of various individual modularized subsystems 30, 40, 50, 60 to be automated based on the node size selected and type of biofuel to be produced and consumed (i.e. biogas or biodiesel).

FIG. 11 shows a non-limiting example of a biogas power generation packaged system formed of pre-fabricated modularized units as described above. The packaged system 100 may include a digester module 130 comprising digester subsystem 30 and ancillary components, biogas treatment module 140 comprising biogas treatment subsystem 40 and ancillary components, biogas storage module 150 comprising biogas storage subsystem 50 and ancillary components, and power generation module 160 comprising electric generation subsystem 60 and ancillary components. Other systems 100 may include more or less pre-fabricated subsystem modules. In some implementations, various subsystems may be provided separately if desired so that an end user may elect to only utilized some of the prefabricated system modules for a given installation. For example, the tankage size requirements may make it impractical to skid mount and transport the size tank needed for biogas storage for a given installation. In such a case, the tankage which comprises biogas storage subsystem 50 may be field fabricated so that a hybrid system of pre-fabricated modules and field fabricated subsystem components may be used.

In one implementation, the power generation module 160 shown in FIG. 10 may be configured to receive a fuel supply twin an external source other than the biogas generated by the packaged biofuel system 100 process, as previously describe herein (see also FIG. 1). Such external sources may include gas supply from a utility company, gas company, or other source. Module 160 may further be configured to capture waste heat from burning the biogas and return at least a portion of the heat to the digester module 130 to enhance the efficiency of the anaerobic waste material digestion process, as already describe herein. The waste heat components may include a platform-mounted heat exchanger 162 (e.g. shell and tube or other) comprising coils 165 configured to extract heat from the hot combustion gases generated by the burning. biogas (see also FIG. 12). The heat exchanger may be a gas-to-air or gas-to-liquid type system, as described herein.

In various implementations, the central control subsystem 80 (see, e.g. FIG. 1) may be incorporated onto one or more of the modules 130, 140, 150, and/or 160, or alternatively may be mounted on a separate dedicated main control module.

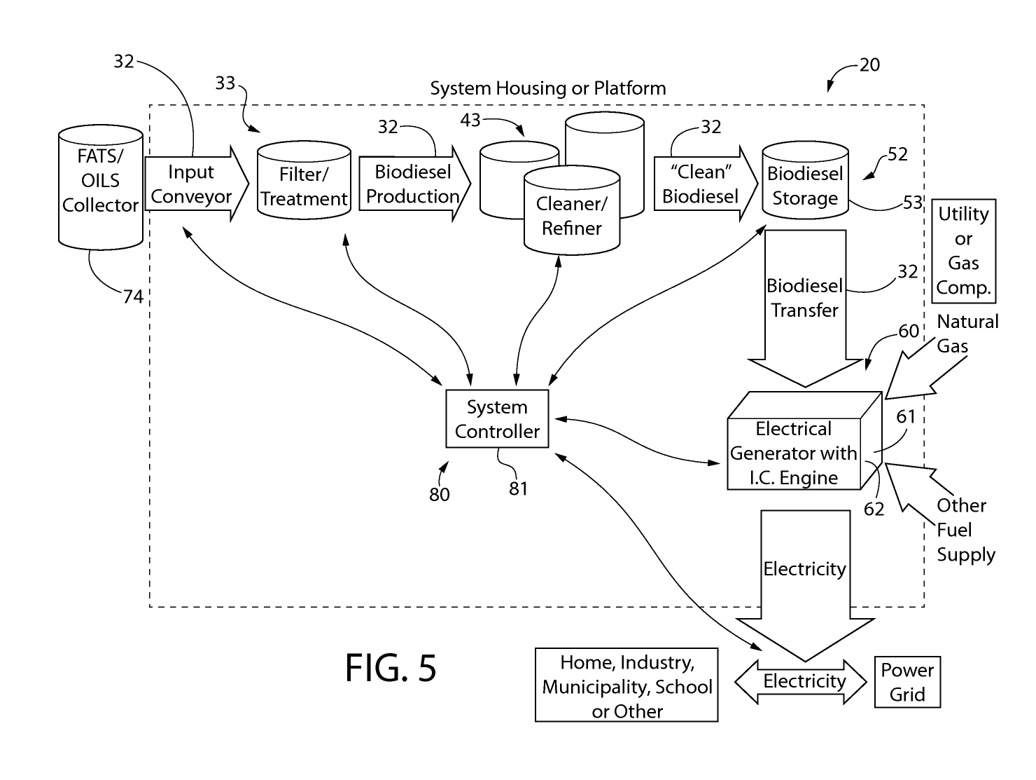

It will be appreciated that a similar modularized system may also be provided and used for a biodiesel power generating facility using the same methodology describe above. Accordingly, with reference to FIG. 5, a modular biodiesel generating system may include a self-supporting filter/treatment module, a self-supporting cleaner/refining module, a self-supporting biodiesel storage module, and a self-supporting power generation module 160 configured to burn biodiesel fuel. These modules are fluidly coupled together in the field at the power generation sue similarly to the modular biogas conversion system described above.

It will be appreciated that for either a modular biogas and biodiesel conversion and power generation system, some or all of the process subsystem modules may be selected and other subsystems that be assembled on site in lieu of being pre-fabricated. Accordingly, some systems may include a combination of factory pre-fabricated subsystem modules and on site erected subsystems for either new or retrofit installations.

A method for forming a biogas conversion system from sub-system modules will now be described with initial reference to FIG. 11. A plurality of factory pre-manufactured different capacity modular subsystem units (e.g. biomass conversion and related process units of capacities 1, 2, 3 or power generation units of capacities 1, 10, 20 KW) are provided each comprising a portable base or platform 161 with components pre-mounted and interconnected thereon. This includes sets of different capacity digester modules 130, biogas treatment modules 140, biogas storage modules 150, and power veneration modules 160 as shown. Other type modules may be provided depending on the nature and configuration of the biofuel power generation system 20 to be assembled. In some instances, the components may be standardized for each module, but not mounted onto an appropriately sized base 161 until selected by a user to allow customization of each module.

A user first generally first selects a type of system to be constructed (e.g. biogas, biodiesel, or both) and then selects a desired electrical output KW size rating based on the quantity of biomass available for conversion and/or other considerations. In this present non-limiting exemplary method, a biogas facility is to be constructed from modular subsystem units. Modules 130, 140, 150, and 160 are selected from the plurality of different size modules available from the factory. The self-supporting portable modules are then transported to the power generation site. The modules are each located at grade or elevated in their desired positions and arrangement of the system. The modules are then interconnected to each other via appropriate interconnecting flow conduits and wiring via the mating interfaces already provided for each module using standardized connection and termination types. In one implementation with a waste heat recovery system as already describe herein, the waste heat flow conduits 64 or 64′ are interconnected in the field between the power generation module 160 and the digester module 130 after placement of these modules.

In the present non-limiting example shown in FIG, 11 a 10 KW biogas conversion plant is selected for installation. The modules 130, 140, and 150 having a capacity (e.g. capacity 2) commensurately sized for a 10 KW power generation module 160 are selected and provided.

In some instances, different sized module instead be selected and integrated into the plant system to serve specific purposes. For example, an end user may select a larger capacity biogas storage module 150 (e.g. capacity 3) and power generation module 160 (e.g. 20KW) if it is anticipated that bioconversion plant may be run on a cyclical operating mode rather than a continuous operating mode depending on the end use for the power generated. Accordingly, various combinations of individual module capacities are possible to create a customized biofuel conversion-power generation system.

Once the desired subsystem modules are selected, the modules may each be lifted as a single self-supporting and self-contained unit for transportation to the power generation site remotely located by a distance from the factory where the prefabricated modules are constructed. The power generation site may be an agricultural or farming she (e.g. crop farm, dairy farm, horse farm, cattle ranch, or other). Non-agricultural sites including municipal waste facilities may be used. In one implementation, the modules 130-150 are sized to allow transport by one or more wheeled vehicles either via the roadways (e.g. tractor trailer), rail (e.g. train rail cars), or combinations thereof.

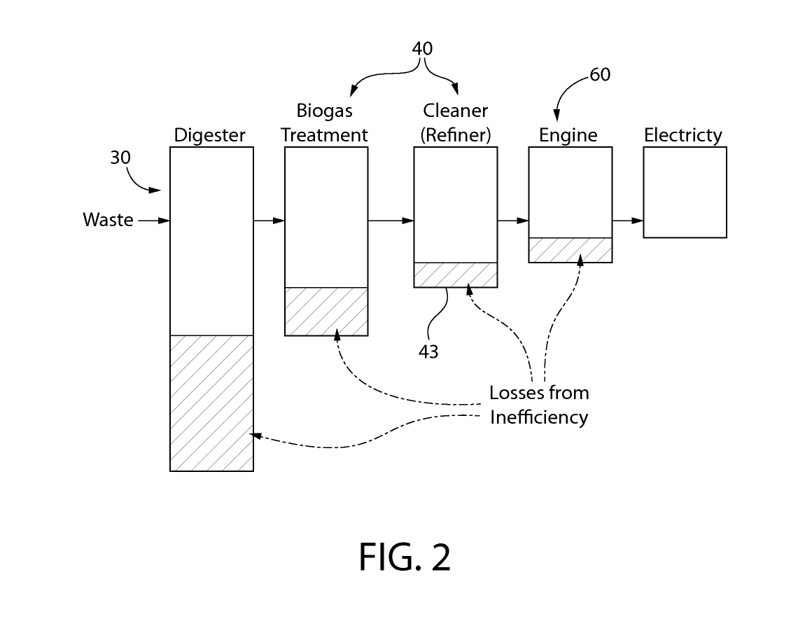

Referring now to FIG. 2, a schematic diagram of an example biogas to electric power generation process is shown. Biodegradable waste (basically any biomass) may be added to the digester 31. A methane (CH₄) mixture may be output from the top combined with carbon dioxide (CO₂), water vapor, and trace gases. Typical output chemistry may be approximately 50-60% CH₄, 20% CO₂, 0-1% O₂ (oxygen) 0-1% N₂ (nitrogen), and 0-1% H₂S (hydrogen sulfide). Fertilizer may be output through the bottom of the digester 31 which is reclaimed for agricultural uses. Water and/or a slurry may be output from the middle of the digester. The size of the digester used may depend on the type of biomass or organic waste material to be digested. For example, cow waste (i.e. manure) from a farm may require a much larger digester than processing used cooking oil from restaurants.

Biogas treatment subsystem 40 treats and purities the methane mixture output from the digester 31 to eliminate some of the less desirable byproducts, such as the oxygen, nitrogen, hydrogen sulfide, and siloxanes. Although an internal combustion engine 61 engine may run with a limited amounts of water, CO₂, nitrogen, and/or H₂S, it may require the elimination of some or most of the byproducts through treatment which may be harmful to equipment and/or seals. The biogas treatment generally involves filtering and/or injection of various chemicals which are mixed with the methane mixture from the digester. The treatment level may depend on location (for regulation purposes and existence of the levels of byproducts). A final treatment stage in the biogas treatment subsystem 40 may include cleaner 43 (refiner).

Referring to FIG. 3, the biofuel power generation system 20 may be configured to direct the biogas output from the biogas treatment subsystem 40 for selective use either (1) as fuel to burn for heating purposes, or (2) to continue to refine the biogas for use in power generation subsystem 60 to produce electricity. A multi-way valve 42 (such as a three-way valve) may be provided to divert the biogas flow from the biogas treatment equipment via suitably configured gas flow conduits (e.g. piping) to either the heating application for direct burning, or to the remainder of the process stream for further refinement and eventual combustion in the engine-generator set 61-62. The central system controller 81 may be configured to control the position of valve 42 and flow path of the biogas.

Because biogas may not be produced at a rate sufficient to direction run the engine-generator set 61-62 regularly or continuously, intermediate storage of the refined biogas in the biogas storage subsystem vessel 51 may be provided. The biogas storage subsystem 51 may include a compressor to keep the biogas stored under pressure in the storage vessel 51. Natural gas from a municipal utility may also be provided to augment the engine fuel needs beyond the production rate of biogas.

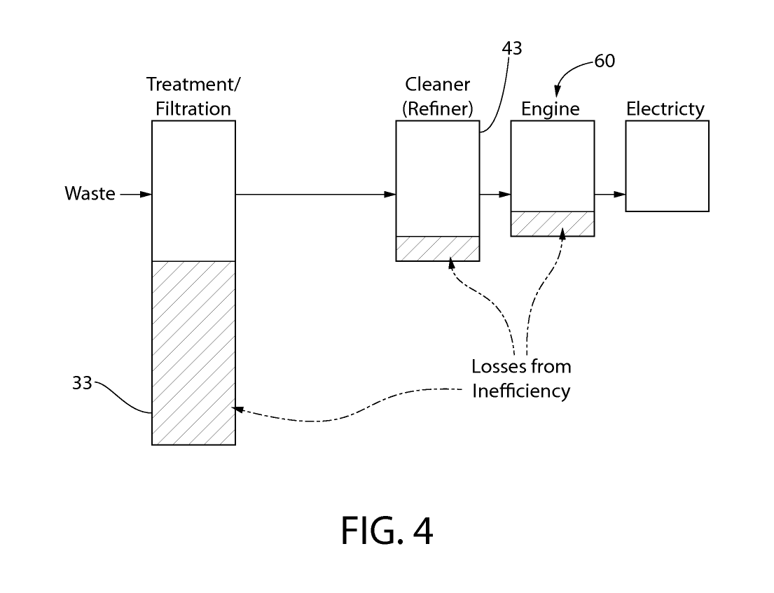

FIG. 4 is a schematic diagram of a biodiesel power generation process. The production of biodiesel may be ideal when the organic waste materials comprise oils (e.g. vegetable and animal fats or oils such as waste cooking oils from restaurants). Treatment and filtration of the biodiesel may be involved and carried out in a biodiesel processing subsystem 33. The filtration may be performed in a series of stages with filters having holes of varying sizes (e.g. between about 0 and 60 microns). The treatment may include a transesterification process conducted via any suitable catalyst or non-catalyst based reaction which reacts lipids in the raw fats and oils with alcohol (e.g. methanol or ethanol) to produce the biodiesel product. The biodiesel may undergo a final processing in a cleaner 43 (refiner) prior to being combusted in engine 60, which in this instance is an internal combustion engine. The refining or purification processes completed in cleaner 43 removes byproducts of the transesterification reaction such as glycerol, soap, excess alcohol, and water. An interim biodiesel storage tank 52 may be provided between the cleaner (refiner) 43 and power generation subsystem 60 for collecting and storing the biodiesel before being burned to produce electricity or dispensed for other on-site or off-site uses. The shaded areas in FIGS. 2 and 4 may illustrate losses that each step may introduce due to the inherent inefficiencies of each system. For example, an internal combustion engine may have an efficiency of approximately 33%. Other variations and examples are possible.

FIG. 5 depicts an example biofuel power generation system 20. In this exemplary configuration, the biofuel generation system may produce biodiesel. The system may include a liquid waste receptacle or collector 74 configured for collecting wastes such as without limitation flits and oils, biodiesel processing subsystem 33, tubular conduits 32, cleaner 43 (refiner), biodiesel storage system 52 which may include fuel tank 53, electric generation subsystem 60 including an internal combustion engine 61 and electrical generator 62, and electronic central control subsystem 80 including a computerized processor-based controller 81. Tubular conduits 32 transport the liquid waste and/or biofuel between the various components shown. In one non-limiting example, the tubular conduits 32 may be piping. Pumps and/or valving may further be included to assist in transferring the liquid waste or biofuel through the system. In some configurations, system 20 may include various components or the full systems shown in both FIGS. 1 and 5.

Referring to FIG. 6, a multi fuel biofuel power generation system is shown comprising provisions for producing and burning biogas and biodiesel to generate electricity. This system includes a biogas production/treatment process train (from FIG. 2) and a biodiesel production/treatment process train (from FIG. 4) fluidly coupled to a single dual fuel internal combustion engine configured for burning either fuel type. The engine-generator set 61-62 of the power generation subsystem 60 produces electricity for end use. Both process trains or paths may be tapped into at any point (such as prior to cleaner/refiner for use with heating or cooking application. The system may additionally be selectively connected with one or more fuel lines from a utility or gas company, or a separate fuel tank. In other systems, no fuel lines or separate fuel tanks may be included. Other variations are possible.

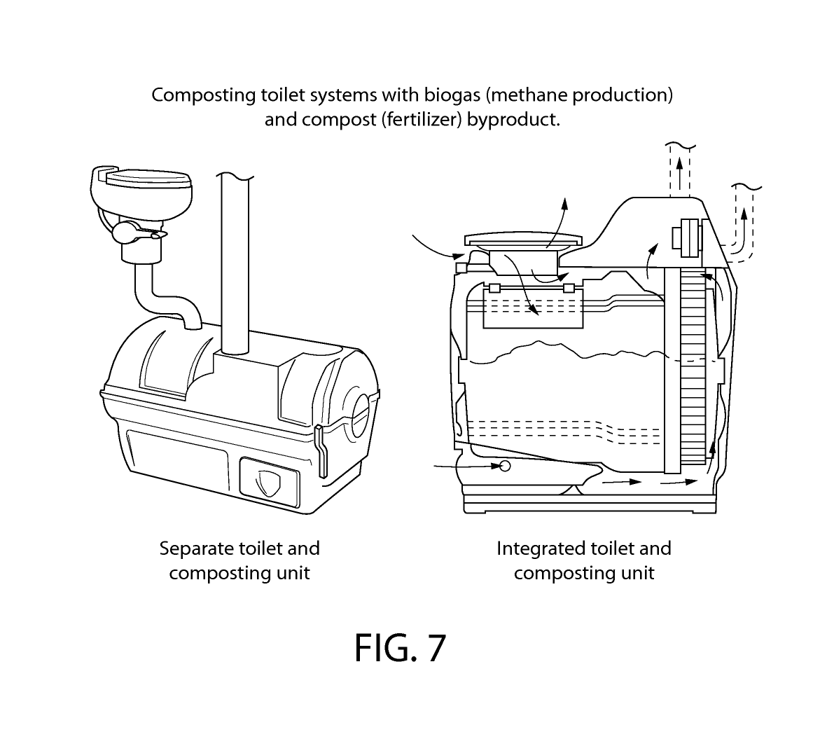

Biogas and useable byproducts front human waste may also be generated, such as by using specialized toilets and processing systems. Examples are shown in FIGS. 7-9 and described below. As one example, a waterless toilet representing a hybrid of an incinerating toilet and a compositing toilet may produce fuel through the decomposition of human waste. Incinerating toilets may quickly handle waste (within 5-30 minutes) and kill all pathogens in the waste, generating inert byproducts. Composting toilets may require little energy and be produced inexpensively, creating a byproduct that can be used as compost. The waterless hybrid toilets may be useful for requiring only a small amount of energy input to create a self-sustaining process which can be completed in a relatively short period of time (such as 1-2 hours). The process may kill all pathogens and oxidize heavy metals and many chemical compounds, leaving the byproducts inert. The produced byproducts may include fuel and biochar, which can be used as compost.

According to another aspect, a biofuel production system may be fully integrated with home waste generation which yields harvestable biogas and fertilizer byproducts for on site use. For example, a toilet may be configured for this purpose to fluidly couple the waste piping to a digester for converting the organic waste material to biogas (i.e. methane), as shown in FIG. 7. The toilet and plumbing could be configured in a number of different ways, including without limitation: dual flush, with the first flush for liquid waste to the city or home sewage system and the second for directing the solid waste to a digester; or single flush all tied/directed to the digester. The entire home waste generation system may be tied together and directed to the digester (e.g. garbage disposal, toilets, etc.). This may he accomplished by forming a piping header or manifold system to fluidly couple the discharge from multiple toilets which is then piped to the digester. The methane generated by the digester may be routed to an engine-generator set 61-62 described herein for producing electricity for home usage. The fertilizer may he used for gardening.

Similar systems may he set up on a much larger scale. For example, in municipalities, towns, cities, villages, etc, may collect and transport sewage, food wastes, animal wastes, or other wastes and provide such waste to the systems shown herein. Municipalities may, for example, pipe sewage directly to facilities or plants set up with systems as shown herein to generate biogas. Municipalities may set up separate trash collection bins and collection vehicles to encourage citizens to dispose of food waste and other biomass separately from other garbage. This waste may be collected by the municipalities and provided as the input material for the systems shown herein. Municipalities may use the biogas generated by these systems as fuel for electric generation systems, and may in turn provide the generated electricity to residents and consumers within the municipality. This may, for example, be used to supplement other power generation facilities within the municipalities. Similarly, private commercial enterprises may similarly employ these systems to gather human and food wastes and use them to generate biogas, which the commercial enterprises may then sell back to municipalities or consumers directly. Many other variations are possible.

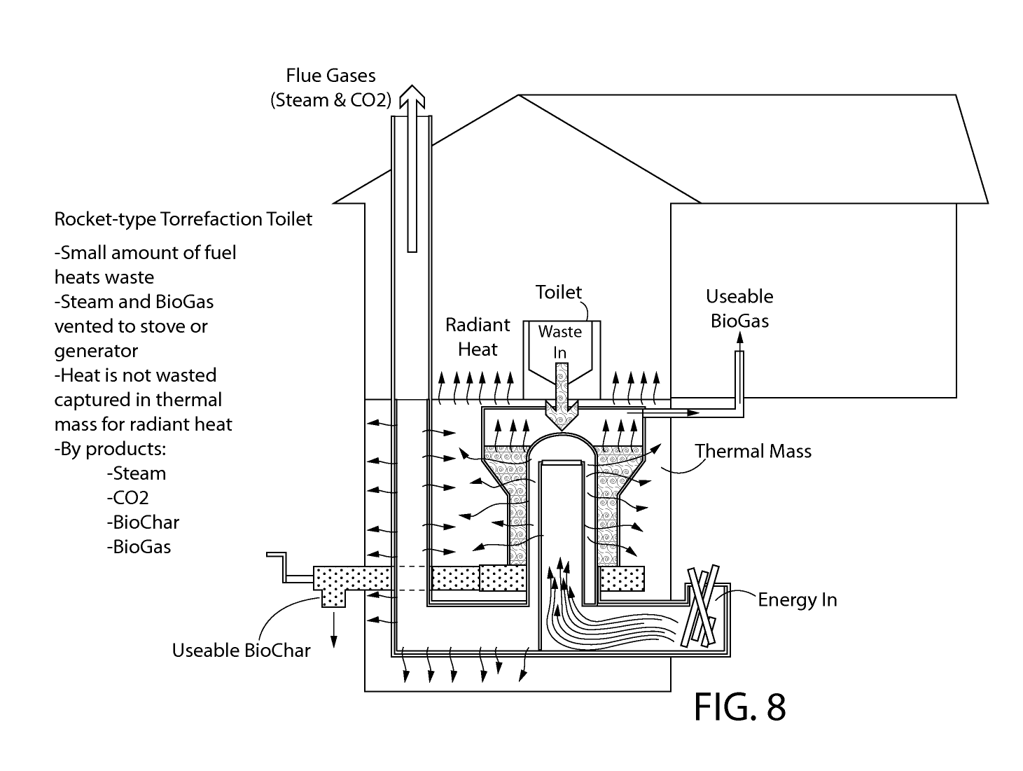

FIG. 8 shows an example rocket-type torrefaction toilet. The rocket-type torrefaction toilet combines aspects of the forgoing organic toilet waste collection system with a torrefaction system that may generate biogas (i.e. methane) and biochar which may be burned.

Torrefaction of biomass may be described as a mild form of pyrolysis at temperatures typically ranging between 200-320° C. An external source of heat may be provided to heat the collected organic toilet waste material for the torrefaction process, such as without limitation burning a combustible solid, liquid, or gaseous form fuel. During torrefaction, the biomass properties are changed to obtain a much better fuel quality for combustion and gasification applications. Torrefaction combined with densification may lead to a very energy dense fuel carrier of 20-25 GJ/ton. Biomass may, in some instances, be an energy source used to create a more sustainable society. However, nature has created a large diversity of biomass with varying specifications. In order to create highly efficient biomass-to-energy chains, torrefaction of biomass in combination with densification (pelletisation/briquetting), may provide a useful step to overcome logistic economics in large scale sustainable energy solutions.

Torrefaction is a thermo chemical treatment of biomass at 200 to 320° C. it is carried out under atmospheric conditions and in the absence of oxygen. During the process, the water contained in the biomass as well as superfluous volatiles are removed, and the biopolymers (cellulose, hemicellulose and lignin) partly decompose giving off various types of volatiles. The final product is the remaining solid, dry, blackened material which is referred to as “torrefied biomass” or “bio-coal”.

During the process, the biomass loses typically 20% of its mass (dry bone basis), while only 10% of the energy content in the biomass is lost. This energy (the volatiles) can he used as a heating fuel for the torrefaction process. After the biomass is torrefied, it can be densified, usually into briquettes or pellets using conventional densification equipment, to further increase the density of the material and to improve its hydrophobic properties.

Torrefaction provides the following potential benefits: Lower energy requirements than pyrolysis, removes moisture and compacts organic material so that it is dense and suitable for later use; renders pathogens inert; condenses bulk of material for storage; and condensed material is hydrophobic allowing for long term storage.

In some alternatives, solar energy may be used as an external source of heat for the torrefaction process. For example, a solar parabolic or other type reflectors may be used to harvest solar energy for the external heat source in lieu of burning fuel. Steam and biogas may be byproducts from these systems, and may be vented to a stove or generator. Additionally, heat may be captured in a thermal mass to create radiant heat. Human biowaste collection/torrifaction modules may be combined with other biogas/biodiesel modules shown herein.

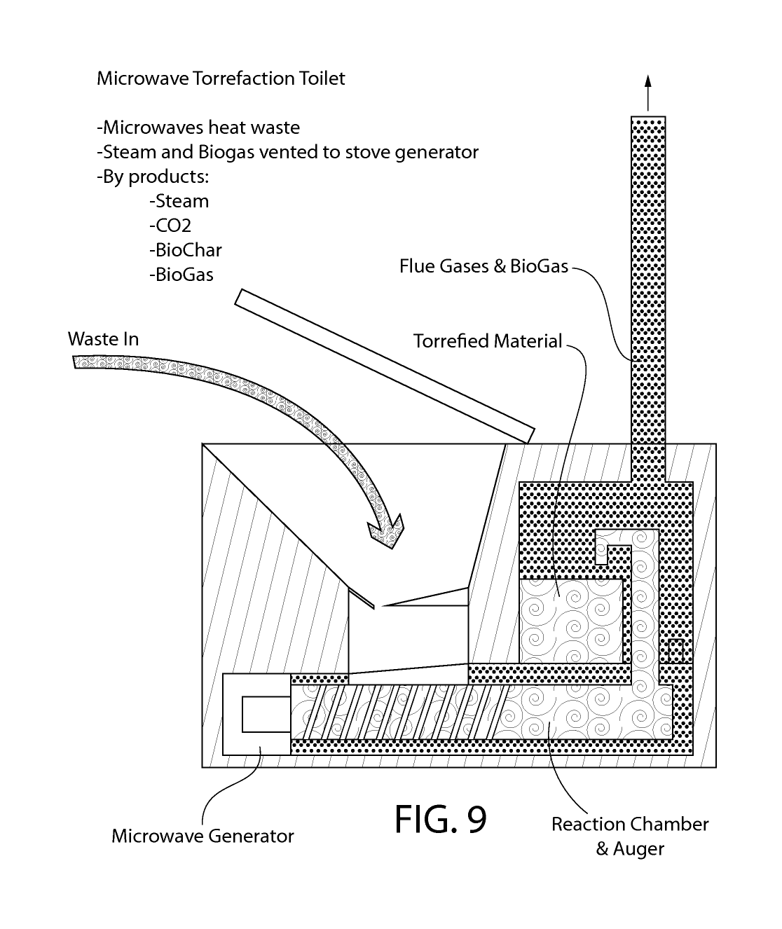

In other alternatives, microwave energy may be used as the external heat source for the torrefaction process. FIG. 9 shows a microwave torrefaction toilet which integrates the toilet and torrefaction process into a single self-contained unit. Waste entering the toilet may be fed to a compartment adjacent a microwave generator. The compartment may be any type of compartment, and may serve as a reaction chamber for microwaves provided by the microwave generator. The microwave generator may heat the waste, creating steam, biogas, CO2, and/or biochar. In some systems, an auger or another device may move the solid byproducts to a separate chamber, creating additional room in the reaction chamber for additional waste. A flue may be connected to vent the biogas from the system, while the terrified matter may reside in a separate container to be used as desired. A controller, such as controller 81, may control many or all aspects of this system. For example, the controller 81 may be connected with a sensor (such as an infrared sensor, weight sensor in the floor, pressure sensor in a toilet seat, or other sensor). The controller may determine when to operate the microwave based on information from the sensor. For example, upon an indication that a person is near the toilet, the controller may prepare the microwave for use. When the controller determines that the person has left the area near the toilet, the controller may operate the microwave to heat the waste. The controller may additionally control the auger or other transportation device to move the heated waste from the reaction chamber, and/or may operate the flue to transport generated biogas to another device (such as a storage container, stove, or engine). Many other variations are possible.

In another system, a urine hydrolysis system may be created to generate biogas from human urine. The system may be integrated with a toilet or be a standalone system. The system may collect urine from a human (or animal), and combine the urine with one or more urea solutions and/or ammonia. The urine may react with the urea solutions and/or ammonia, and may generate biogas, which in turn may be used to generate electricity. In some variations, a solar panel may be integrated with the urine hydrolysis system. Other variations are possible.

Biochar or Terra preta may be charcoal created by pyrolysis of biomass. A primary use of biochar may be for carbon sequestration and bio-energy with carbon capture and storage. Biochar may be used to improve water quality, increase soil fertility, raise agricultural productivity, and/or reduce pressure on old-growth forests. Biochar is able to attract and hold moisture, nutrients (like nitrogen and phosphorous), and agrochemicals. Biochar can be used to enhance crop yields, enrich soil and protect water, and as a carbon negative. As such, the systems described herein which generate biochar may, in some instances, have biochar distribution components which may transfer biochar to the soil or package the biochar into quantities for use with improving water quality or for distribution into the soil.

Any of the systems herein may be used with prime power applications, with the generated biogas operating as the primary fuel source for the electric generation, or operating as one of a number of fuel sources such as in systems where the biogas is augmented or supplemented with utility natural gas supply. Any of the systems herein may alternatively he used with standby power applications. In standby power applications, the biogas may, in some situations, constitute the entirety or majority of power needs, and be augmented only by utility natural gas occasionally (or not at all). Many other variations are possible.

While the foregoing description and drawings represent some example systems, it will be understood that various additions, modifications and substitutions may be made therein without departing from the spirit and scope and range of equivalents of the accompanying claims. In particular, it will be clear to those skilled in the art that the present invention may be embodied in other forms, structures, arrangements, proportions, sizes, and with other elements, materials, and components, without departing from the spirit or essential characteristics thereof. In addition, numerous variations in the methods/processes. One skilled in the art will further appreciate that the invention may be used with many modifications of structure, arrangement, proportions, sizes, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being defined by the appended claims and equivalents thereof, and not limited to the foregoing description or embodiments. Rather, the appended claims should be construed broadly, to include other variants and embodiments of the invention, which may he made by those skilled in the art without departing from the scope and range of equivalents of the invention.

What is claimed is:

1. A modular integrated biofuel power generation system comprising:
a digester subsystem module including a digester configured to convert biomass into biogas; and
an electric power generation subsystem module including an internal combustion engine and electrical generator configured for generating electricity, the internal combustion engine fluidly coupled to and configured to burn the biogas produced by the digester;
the components of each module being mounted on individual self-supporting and transportable structural platforms;
Wherein the modules are fluidly coupled together on a power generation site after placement of each module.

2. The system of claim 1, wherein fluid and electrical interconnection between components in each module are made in a controlled factory environment before shipment of the modules to the site.

3. The system of claim 1, further comprising a waste heat recovery system configured to capture heat generated by the internal combustion engine of the power generation subsystem module.

4. The system of claim 3, wherein the waste heat recovery system includes an insulated flow loop or path configured to transfer a heated working fluid from the power generation subsystem module to the digester subsystem module for heating the digester.

5. The system of claim 4, wherein the waste heat recovery system is a once-through hot air type system including a blower.

6. The system of claim 4, wherein the waste heat recovery system is a recirculating coolant type system including a pump.

7. The system of claim 3, wherein the waste heat recovery system is configured to extract heat from combustion gases generated by the internal combustion engine.

8. The system of claim 7, wherein the platforms of the modules are configured to fit onto a flatbed truck for transport

9. The system of claim 1, further comprising a self-supporting biogas treatment subsystem module interconnected on site between the digester subsystem module and the power generation subsystem module.

10. The system of claim 1, further comprising a self-supporting biogas storage subsystem module interconnected on site between the digester subsystem module and the power generation subsystem module.

11. The system of claim 1, further comprising a self-supporting central system controller subsystem module operably interconnected to the power generation and digester subsystem modules, the controller subsystem module controlling operation of the digester and the internal combustion engine.

12. The system of claim 1, wherein the internal combustion engine is configured to burn fuel from an alternate non-biogas fuel source.

13. The system of claim 12, wherein the internal combustion engine is dual fuel engine configured to burn biogas and liquid biodiesel fuels.

14. The system of claim 1, wherein the biomass comprises manure.

15. A modular integrated biofuel power generation system comprising
a filter/treatment subsystem module configured to convert raw oils and fats into biodiesel via a chemical reaction;
a cleaner/refining subsystem module configured to purify the biodiesel by removing byproducts of the chemical reaction; and
a power generation subsystem module including an internal combustion engine and electrical generator configured for generating electricity, the internal combustion engine fluidly coupled to and configured to burn the biodiesel produced by the system;
the components of each module being mounted on an individual self-supporting and transportable structural platforms;
wherein the modules are fluidly coupled together on a power generation site after placement of each module.

16. The system of claim 15, further comprising a self-supporting biodiesel storage module interconnected on site between the cleaner/refining subsystem module and the power generation subsystem module.

17. The system of claim 15, further comprising a self-supporting central system controller subsystem module operably interconnected to the power generation and filter/treatment subsystem modules, the controller subsystem module controlling operation of the filter/treatment and the internal combustion engine.

18. The system of claim 15, wherein the internal combustion engine is dual fuel engine configured to burn biogas and liquid biodiesel fuels.

19. A method for forming a modular integrated biofuel power generation system comprising:
selecting a factory prefabricated self-supporting first digester subsystem module having a first capacity from a plurality of prefabricated self-supporting digester subsystem modules each having a different capacity, the first digester subsystem module including a digester configured to convert biomass into biogas, wherein the first digester subsystem module comprises a transportable structural equipment platform configured to be lifted as a single unit;
selecting a factory prefabricated self-supporting first biogas treatment subsystem module having a first capacity from a plurality of prefabricated self-supporting biogas treatment subsystem modules each having a different capacity the first biogas treatment subsystem module configured to refine the biogas produced by the digester, wherein the first biogas treatment subsystem module comprises a transportable structural equipment platform configured to be lifted as a single unit;
selecting a factory prefabricated self-supporting first electric power generation subsystem module haying a first electrical output rating from a plurality of prefabricated self-supporting power generation subsystem modules each having a different electrical output rating, the first electric power generation subsystem module including an internal combustion engine and electrical generator configured for generating electricity, wherein the first power generation subsystem module comprises a transportable structural equipment platform configured to be lifted as a single unit;
transporting the first digester subsystem module, first biogas treatment subsystem module, and first electric power generation subsystem module from a factory to a remote power generation site location;
emplacing the first digester subsystem module, first biogas treatment subsystem module, and first electric power generation subsystem module at the power generation site;
fluidly coupling the digester to the first biogas treatment subsystem module via a field run flow conduit; and
fluidly coupling the internal combustion engine to the first biogas treatment subsystem module via a field run flow conduit.

20. The method of claim 19, further comprising;
a waste heat recovery system configured to capture heat generated by the internal combustion engine of the power generation subsystem module; and
installing an insulated flow loop or path at the power generation site configured to transfer a heated working fluid from the first power generation subsystem module to the first digester subsystem module for heating the digester.
